(19) United States
(12) Patent Application Publication
(10) Pub. No.: US 2014/0120568 A1
(43) Pub. Date: May 1, 2014

(54) SYSTEM AND METHODOLOGY FOR ANALYZING LOW-DENSITY LIPOPROTEIN TRANSPORT WITHIN A MULTI-LAYERED ARTERIAL WALL

(71) Applicant: Kambix Innovations, LLC, Albuquerque, NM (US)

(72) Inventors: Kambiz Vafai, Mission Viejo, CA (US); Stephen Chung, Westminster, CA (US)

(73) Assignee: Kambix Innovations, LLC, Albuquerque, NM (US)

(21) Appl. No.: 14/060,022

(22) Filed: Oct. 22, 2013

Related U.S. Application Data
(60) Provisional application No. 61/718,849, filed on Oct. 26, 2012.

(51) Int. Cl. G01N 33/50

(52) U.S. Cl. CPC G01N 33/5088
USPC 435/29

(57) ABSTRACT

Low-density lipoprotein (LDL) transport while incorporating the thickening of the arterial wall and cholesterol lipid accumulation can be analyzed. A multi-layered model can be adopted to represent the heterogeneity using the Darcy-Brinkman and Staverman filtration equations to describe transport within the porous layers of the wall. The fiber matrix model can be utilized to represent the cholesterol lipid accumulation and the resulting variable properties. The impact of atherosclerotic wall thickening is shown to be negligible in the axial direction, but is found to be considerable in the radial direction within intima. The reference values of intima's porosity and effective fiber radius are obtained through the fiber matrix model, which characterizes the micro-structure within the intima.

### CROSS-REFERENCE TO PROVISIONAL PATENT APPLICATION

This patent application claims the benefit under 35 U.S.C. §119(e) of U.S. Provisional Application Ser. No. 61/718,849 entitled “Low-Density Lipoprotein Transport within a Multi-Layered Arterial Wall-Effect of the Atherosclerotic Plaque/Stenosis,” which was filed on Oct. 26, 2012 and is incorporated herein by reference in its entirety.

### FIELD OF THE INVENTION

Embodiments are generally related to a cardiovascular system. Embodiments also relate to a system and method for analyzing low-density lipoprotein transport within a multi-layered arterial wall. Embodiments are additionally related to a system and method for analyzing the impact of atherosclerotic plaque/stenosis on LDL transport.

### BACKGROUND

Cardiovascular disease is a critical issue with respect to human health due to the high rate of death that it causes. Almost 80 million adults in America have one or two types of cardiovascular diseases (American Heart Association, 2007; Khakpour and Vafai, 2008). Atherosclerosis is a type of cardiovascular disease that usually occurs in a larger artery like aorta and leads to other types of cardiovascular diseases. This aortic disease itself is the 14 cause of death in America (Gillum, 1995; Khanafer et al. 2009) with the subsequent mortality rate increasing by 1-2% per hour after it is discovered (Wang and Dake, 2006; Khanafer and Berguer 2009). Almost half a trillion dollars were spent on health care associated with the cardiovascular diseases in 2008 in the United States (American Heart Association, 2008; Hossain et al., 2011). Clearly, this figure is higher today.

Although the main cause of atherosclerosis is still not fully established, low-density lipoprotein (LDL) is considered to be one of the main factors in causing atherosclerosis. LDL oxidized with free radicals inside the arterial wall damages the cells and compromises the immune response resulting in a dysfunction within the arterial wall and plaque formation thus narrowing the available cross section for lumen flow. In most of the cases, the first symptom of atherosclerosis is a heart attack and half of these lead to death. On an annual basis, 1.1 million Americans die from atherosclerosis complications, which accounts for ⅕ of deaths in the United States (American Heart Association, 2005, 2006; Hossain et al., 2011). Therefore, better understanding of the formation of atherosclerosis and stenosis can lead to a better diagnosis and treatment of this disease.

Starting with lipid accumulation, atherosclerosis results a lipid filled plaque that can block blood flow through an artery. Three stages can be cited during development of atherosclerosis such as cholesterol lipid accumulation inside arterial wall, especially within the intima layer, thickening of the wall due to component deposits that cause stenosis and dysfunction of endothelium and fibrous cap formed on the inner wall surface within endothelium and intima (Hossain et al., 2011). Likewise, stenosis can be classified into three grades (Buchanan and Kleinstreuer, 1997) such as no stenosis, moderate stenosis, and severe stenosis (Ai and Vafai, 2006). Ai and Vafai (2006) had discussed the LDL transport and its deposition within the arterial wall along with variations in its thickness due to plaque formation.

A comprehensive model of LDL accumulation within the arterial wall is crucial in better understanding of the involved processes leading to atherosclerosis. The arterial wall is actually composed of glycocalyx, endothelium, intima, internal elastic lamina (IEL), media, and adventitia, with different hydraulic and mass transport properties. Transport within these layers have been investigated, both from macro-scale view point (Huang et al., 1994; Tada and Tarbell, 2004; Prosi et al., 2005; Ai and Vafai, 2006) as well as a micro-scale point of view (Curry, 1984a, b; Fry, 1985; Wen et al., 1988; Huang et al., 1992; Huang et al., 1997; Huang and Tarbell, 1997; Yuan et al., 1991; Weinbaum et al., 1992; Karner et al., 2001; Liu et al., 2011; Chung and Vafai, 2012). For example, Ai and Vafai (2006) utilized a reverse procedure to solve for hydraulic permeability, effective diffusivity, and reflection coefficient of arterial porous layers using a circuit analogy. On the other hand, Huang et al. (1994); Karner et al. (2001); Liu et al. (2011), and Chung and Vafai (2012) obtained the properties based on the micro-structure information using the pore theorem and fiber matrix model.

A number of works (Huang et al., 1994; Karner et al., 2001; Liu et al., 2011) indicate that the arterial transport properties are controlled by the microstructure in each of the different layers of the arterial wall. Several theorems were introduced to enable calculation of the properties based on the parameters that describe the microstructure, such as fiber matrix model for obtaining the properties within the intima layer. However, these focus on transport inside a normal healthy artery only, instead of that under initiation or development of atherosclerosis.

A multi-layered model (Ai and Vafai, 2006; Yang and Vafai, 2006, 2008) accurately represents the layered structure with different transport behavior within each of the layers. These layers are endothelium, intima, IEL, and media, where the Staverman-Keden-Katchalsky membrane equation (Kedem and Katchalsky, 1958) is invoked to describe the mass convection inside a low permeability porous medium. The impact of macro-structure such as stenosis (Ai and Vafai, 2006; Kanafer et al., 2009) or bifurcation (Khakpour and Vafai, 2008) has been studied by several scientists. However, the macro-structure might not play a significant role and as an example, in Ai and Vafai's study (2006), the effect of stenosis on LDL transport was not found to be pronounced. On the other hand, due to atherosclerosis, the fibrous cap and lipid core formed by the hyperplasia of arterial cells and fibers and accumulation of cholesterol lipid inside the arterial wall impacts the microstructure, and further affects the transport properties.

In this study based on the multi-layered model, the impact due to changes in the microstructure which results in a variation of transport properties is analyzed comprehensively while studying the effect of atherosclerosis on arterial transport.

Therefore, a need exists for an improved system and method for comprehensively analyzing the impact due to changes in the microstructure which results in a variation of transport properties while studying the effect of atherosclerosis on arterial transport.

### SUMMARY

The following summary is provided to facilitate an understanding of some of the innovative features unique to the disclosed embodiment and is not intended to be a full description. A full appreciation of the various aspects of the embodiments disclosed herein can be gained by taking the entire specification, claims, drawings, and abstract as a whole.

It is another aspect of the disclosed embodiments to provide for a method and system for analyzing low-density lipoprotein transport within a multi-layered arterial wall.

It is a further aspect of the disclosed embodiments to provide for a method and system for analyzing the impact of atherosclerotic plaque/stenosis on LDL transport.

The aforementioned aspects and other objectives and advantages can now be achieved as described herein. Low-density lipoprotein (LDL) transport while incorporating the thickening of the arterial wall and cholesterol lipid accumulation is analyzed. A multi-layered model is adopted to represent the heterogeneity using the Darcy-Brinkman and Staverman filtration equations to describe transport within the porous layers of the wall. The fiber matrix model is utilized to represent the cholesterol lipid accumulation and the resulting variable properties. The impact of atherosclerotic wall thickening is shown to be negligible in the axial direction, but is found to be considerable in the radial direction within intima. The reference values of intima's porosity and effective fiber radius are obtained through the fiber matrix model, which characterizes the micro-structure within the intima. Transport through dysfunctional endothelium and fibrous cap, and the impact on hydraulic and molecular transport properties by LDL accumulation in a thickened arterial wall is analyzed. The effect of variable properties on plasma and LDL molecular transport is also discussed.

The disclosed embodiments can be utilized for analyzing the impact of atherosclerotic plaque on LDL transport. In addition to considering the macro-structure effect, the micro-structure variation due to molecular accumulation and its effect on LDL transport is also analyzed. The impact of stenosis formation, thickening of intima, and transport properties variations due to LDL accumulation associated with atherosclerosis, as well as consideration of the dysfunctional endothelium and fibrous cap, is investigated through an advanced model.

The microstructure details and characteristics of the endothelium and intima due to the formation of plaque/stenosis can be incorporated with respect to particular embodiments. Pertinent scenarios for transport through a dysfunctional endothelium and fibrous cap within intima are invoked. The variable intima properties affected by LDL molecule accumulation are analyzed, and its impact on the hydraulic and molecular transport in a thickened arterial wall is examined. Lower porosity by lipid blockage results in a lower permeability, which is diminished by thickening of effective fiber due to more space between the fibers as a result of stenosis.

It is to be understood that both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments and together with the description serve to explain the principles of the embodiments.

### BRIEF DESCRIPTION OF THE FIGURES

The accompanying figures, in which like reference numerals refer to identical or functionally-similar elements throughout the separate views and which are incorporated in and form a part of the specification, further illustrate the disclosed embodiments and, together with the detailed description of the invention, serve to explain the principles of the disclosed embodiments.

FIGS. 1A and 1B illustrate schematic diagrams of configuration for endothelium, intima, IEL, and intima fiber matrix and arterial wall respectively, in accordance with the disclosed embodiments;

FIG. 1C illustrates schematic diagram of configuration for analyzed domain for a healthy artery (Yang and Vafai, 2006; Ai and Vafai, 2006), in accordance with the disclosed embodiments;

FIG. 2A illustrates schematic diagram of configuration for analyzed domain in the presence of stenosis (Ai and Vafai, 2006), in accordance with the disclosed embodiments;

FIGS. 2B and 2C illustrate schematic diagrams of configuration for proteoglycan fibers within intima (Huang et al. 1994) and fiber matrix filled with cholesterol lipid respectively, in accordance with the disclosed embodiments;

FIG. 2D illustrates schematic diagram of configuration for analyzed region for the stenosis/plaque case, in accordance with the disclosed embodiments;

FIGS. 3A-3B illustrate graphs showing a normalized LDL concentration c/c₀ across diseased artery layers in the presence of stenosis with δ=0.5 and x₀=2R₀ for comparison with Ai and Vafai (2006) work, in accordance with the disclosed embodiments;

FIGS. 3C-3D illustrate graphs showing a normalized LDL concentration c/c₀ across diseased artery layers in the presence of stenosis with δ=0.5 and x₀=2R₀ for two different boundary conditions at the media-adventitia interface (r=3.134 mm) c=0 and c=0.012c₀, in accordance with the disclosed embodiments;

FIG. 4 illustrates a graph showing normalized LDL concentration c/c₀ across diseased artery layers in the presence of stenosis at center of the plaque (x=xₛₜ; x₀=2R₀) with different wall thickening δ and location of stenosis xₛₜ, in accordance with the disclosed embodiments;

FIGS. 5A-5B illustrate graphs showing filtration velocity along the lumen-endothelium interface for different wall thickening ratio δ and stenosis locations xₛₜ; and different values of the plaque half widths x₀, respectively, in accordance with the disclosed embodiments;

FIGS. 5C-5D illustrate graphs showing LDL concentration along the lumen-endothelium interface for different wall thickening ratio δ and stenosis locations xₛₜ; and different values of the plaque half widths x₀, respectively, in accordance with the disclosed embodiments;

FIGS. 6A-6B illustrate graphs showing filtration velocity along the intima-IEL interface with a) different plaque half widths x₀ and reversed boundary conditions (inlet: pressure, outlet: velocity) respectively, in accordance with the disclosed embodiments;

FIGS. 6C-6D illustrate graphs showing LDL concentration along the intima-IEL interface with a) different plaque half widths x₀ and reversed boundary conditions (inlet: pressure, outlet: velocity) respectively, in accordance with the disclosed embodiments;

FIGS. 7A-7B illustrate graphs showing normalized LDL concentration c/c₀ across IEL and media of a diseased artery at different locations x in the presence of stenosis with δ=0.5, xₛₜ=5.58 cm and 16.74 cm for boundary condition at the media-adventitia interface (r=3.314 mm) as ∂c/∂r=0, in accordance with the disclosed embodiments;

FIGS. 7C-7D illustrate graphs showing normalized LDL concentration c/c₀ across IEL and media of a diseased artery at different locations x in the presence of stenosis with δ=0.5, xₛₜ=5.58 cm and 16.74 cm for boundary condition at the media-adventitia interface (r=3.314 mm) as c=0, in accordance with the disclosed embodiments;

FIGS. 7E-7F illustrate graphs showing normalized LDL concentration c/c₀ across IEL and media of a diseased artery at different locations x in the presence of stenosis with δ=0.5, xₛₜ=5.58 cm and 16.74 cm for boundary condition at the media-adventitia interface (r=3.314 mm) as c=0.012c₀, in accordance with the disclosed embodiments;

FIGS. 8A-8B illustrate graphs showing filtration velocity and LDL concentration across endothelium, fibrous cap, and intima for different physical attributes of endothelium and fibrous cap given in Table 2b, respectively, in accordance with the disclosed embodiments;

FIGS. 9A-9B illustrate graphs showing filtration velocity along the intima-IEL interface. Intima properties were obtained through fiber matrix model with protein fiber radius r_{F} of 2.31 nm and variations of intima porosity, ε or (ε_{PG}, ε_{CG}) based on data given in Table 3a and variations of intima porosity ε and effective protein fiber radius r_{f} based on data given in Table 3b, and compared with those based on the properties obtained by Ai and Vafai (2006) and Liu et al (2011) given in Table 3c, respectively, in accordance with the disclosed embodiments;

FIGS. 9C-9D illustrate graphs showing LDL concentration along the intima-IEL interface. Intima properties were obtained through fiber matrix model with protein fiber radius r_{f} of 2.31 nm and variations of intima porosity, ε or (ε_{PG}, ε_{CG}) based on data given in Table 3a and variations of intima porosity ε and effective protein fiber radius r_{f} based on data given in Table 3b, and compared with those based on the properties obtained by Ai and Vafai (2006) and Liu et al (2011) given in Table 3c, respectively, in accordance with the disclosed embodiments;

FIGS. 10A-10B illustrate graphs showing effect of variations in the effective porosity ε_{Lip} and protein fiber thickening ratio r_{f,Lip}/r_{f} on the filtration velocity across a diseased arterial wall, in accordance with the disclosed embodiments; and

FIGS. 10C-10D illustrate graphs showing effect of variations in the effective porosity ε_{Lip} and protein fiber thickening ratio r_{f,Lip}/r_{f} on the LDL molecule concentration across a diseased arterial wall, in accordance with the disclosed embodiments.

### DETAILED DESCRIPTION

The particular values and configurations discussed in these non-limiting examples can be varied and are cited merely to illustrate at least one embodiment and are not intended to limit the scope thereof.

The following Table 1 provides the various symbols and meanings used in this section:

TABLE 1

| c | LDL concentration | v | filtration (radial) velocity |
|---|---|---|---|
| D | LDL diffusivity | x | axial location from inlet |
| G | Kozney constant | X₀ | half width of atherosclerotic |
| | | | plaque |
| H | thickness of the layers | xₛₜ | axial location of atherosclerotic |
| | | | plaque/stenosis |
| k | reaction coefficient | α | length ratio of proteoglycan |
| | | | monomers to central filament |
| K | hydraulic permeability | β | length ratio of |
| | | | glycosaminoglycan fiber to |
| | | | protein core |
| L | length of the artery | δ | ratio of maximum thickness of |
| | | | plaque to radius of lumen |
| p | hydraulic pressure | ε | porosity |
| r | radial location from the | φ | partition coefficient |
| | centerline | | |
| rₘ | molecular radius | μ | viscosity |
| r_{CF} | radius of central filament | σ | reflection coefficient |
| r_{CP} | radius of proteoglycan core | Δ | distance between protein fibers |
| | protein | | |
| r_{f} | effective intima fiber radius | Subscripts | |
| r_{G} | radius of glycosaminoglycan | 0 | refers to entrance condition |
| r_{M} | effective monomer radius | eff | refers to effective property |
| R₀ | radius of lumen domain | f | refers to plasma property |
| u | axial velocity | PG | refers to proteoglycan |
| ū | velocity vector | CG | refers to collagen |
| U₀ | maximum velocity at entrance | Lip | refers to property affected by |
| | | | LDL lipid accumulation |

In following Table 2: a) Hydraulic and LDL transport properties for each of the layers/domains (Ai and Vafai, 2006; Chung and Vafai, 2012); b) Properties obtained in previous works for dysfunctional endothelium and fibrous cap.

TABLE 2
a

| a) | Lumen | Endothelium | Intima | IEL | Media |
|---|---|---|---|---|---|
| Diffusivity | 2.87 × 10⁻¹¹ | 8.154 × 10⁻¹⁷ | 5 × 10⁻¹² | 3.18 × 10⁻¹⁵ | 5 × 10⁻¹⁴ |
| D_{eff} [m²/s] | | | | | |
| Permeability | | 3.2172 × 10⁻²¹ | 2.2 × 10⁻¹⁶ | 3.2188 × 10⁻¹⁹ | 2 × 10⁻¹⁸ |
| K [m²] | | | | | |
| Refection | | 0.9886 | 0.8292 | 0.8295 | 0.8660 |
| coefficient σ | | | | | |
| Thickness | 3100 | 2 | 10 | 2 | 200 |
| H [μm] | | | | | |
| Viscosity | 3.5 × 10⁻³ | 0.72 × 10⁻³ | 0.72 × 10⁻³ | 0.72 × 10⁻³ | 0.72 × 10⁻³ |
| μ_{eff} [kg/m · s] | | | | | |

b

| b) | Normal endothelium | | Leaky endothelium | | Fibrous cap |
|---|---|---|---|---|---|
| Thickness | 2 μm | | | | 65 μm |
| Hydraulic | 3.21 × 10⁻²¹ | m² | 2.62 × 10⁻¹⁹ | m² | — |
| Permeability | | | | | |
| Effective | 8.15 × 10⁻¹⁷ | m²/s | 1.142 × 10⁻¹⁴ | m²/s | 4.5 × 10⁻¹³ m²/s |
| Diffusivity | | | | | |

| | | | |
|---|---|---|---|
| Reflection | 0.9886 | 0.7240 | — |
| Coefficient | | | |
| Reference | Ai and Vafai (2006) | Curry (1984a, b) | Hossain et al. (2011) |
| | | Chung and Vafai (2012) | |

In following Table 3: Intima properties a) obtained using fiber matrix method with protein fiber radius r₁ of 2.31 nm (Equation 5) and variation of intima porosity, ε or (ε_{PG}, ε_{CG}) given in previous work (Yang and Vafai, 2006; Dabagh et al., 2009); b) obtained using fiber matrix method variations in both intima porosity ε (Dabagh et al., 2009, ε=ε_{PG}ε_{CG}) and protein fiber radius r_{f}; c) obtained in the prior works (Ai and Vafai, 2006; Liu et al., 2011).

TABLE 3

| | | | | | |
|---|---|---|---|---|---|
| | Porosity | Effective | | | |
| | ε or (ε_{PG}, | fiber | Permeability | Diffusivity | Reflection |
| # | ε_{CG}) | radius [nm] | K [m²] | D_{eff} [m²/s] | coefficient σ |

a)

| | | | | | |
|---|---|---|---|---|---|
| 1 | 0.983 | 2.31 | 1.66 × 10⁻¹⁶ | 1.35 × 10⁻¹¹ | 0.1771 |
| 2 | (0.9568, | 2.31 | 3.93 × 10⁻¹⁷ | 3.7 × 10⁻¹² | 0.7983 |
| | 0.8387) | | | | |
| 3 | (0.9866, 0.95) | 2.31 | 2.1 × 10⁻¹⁶ | 9.7 × 10⁻¹² | 0.3247 |
| 4 | 0.9373 | 2.31 | 2.5 × 10⁻¹⁷ | 6.78 × 10⁻¹² | 0.7512 |

b)

| | | | | | |
|---|---|---|---|---|---|
| 1 | 0.9373 | 2.08 | 2.02 × 10⁻¹⁷ | 5.94 × 10⁻¹² | 0.8292 |
| 2 | 0.8025 | 4.17 | 9.59 × 10⁻¹⁸ | 5.7 × 10⁻¹² | 0.8292 |

c)

| | | | | | |
|---|---|---|---|---|---|
| Ai & Vafai | 0.96 | — | 2.2 × 10⁻¹⁶ | 5 × 10⁻¹² | 0.8292 |
| (2006) | | | | | |
| Liu et al. | (0.9568, | — | 4.2 × 10⁻¹⁷ | 3.7 × 10⁻¹²* | 0.7983 |
| (2011) | 0.8387) | | | | |

### 1. FORMULATION

### 1.1. Multi-Layer Model

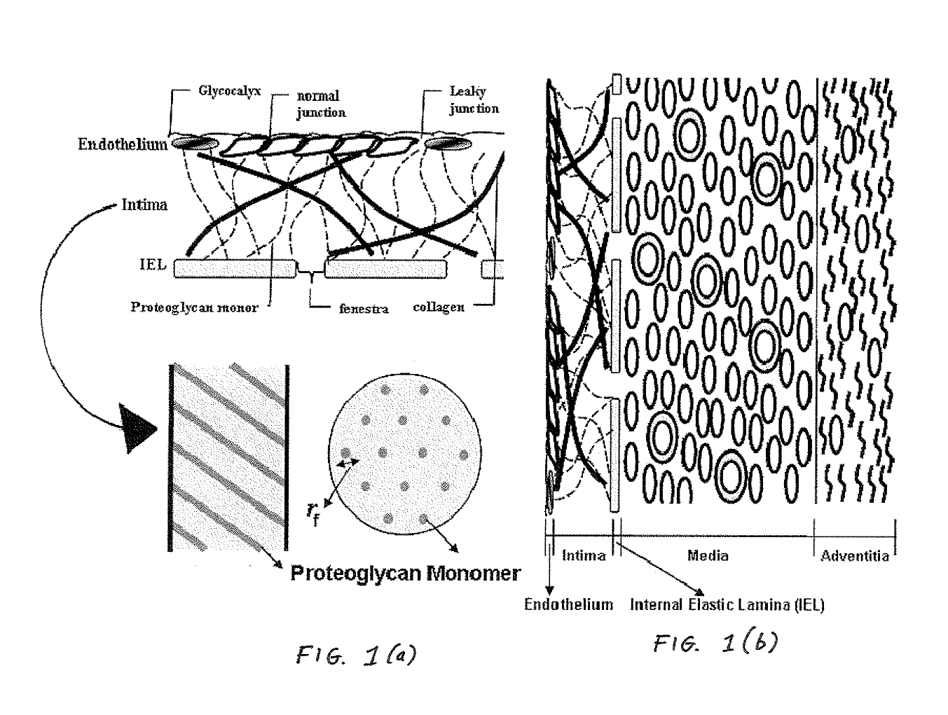

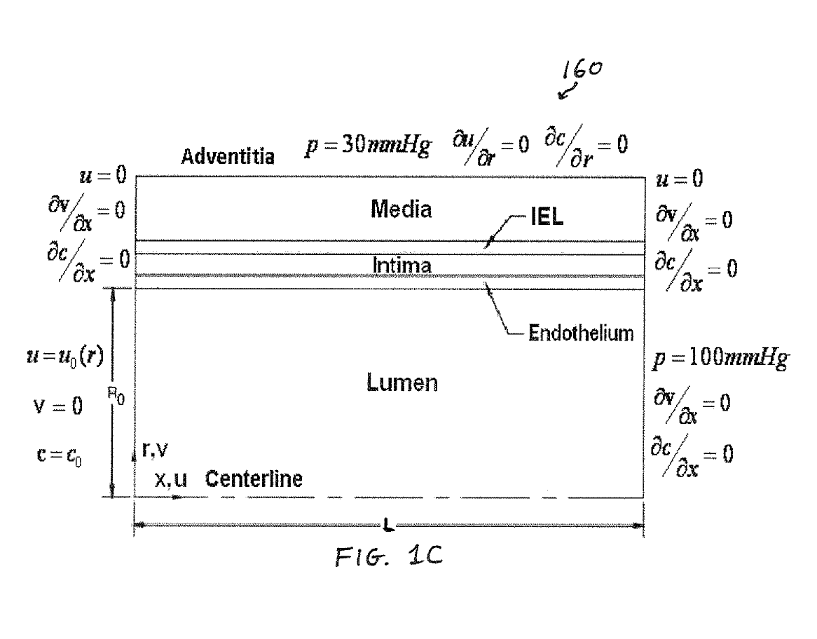

The layered structure of the wall for an artery is shown in FIGS. 1A-1B, from inner to the outer side, have lumen, glycocalyx, endothelium, intima, IEL, media, and adventitia. Glycocalyx is neglected due to its negligible thickness (Michel and Curry, 1999; Tarbell, 2003), and adventitia is embedded into the boundary condition on the outer surface of the wall due to its low resistance (Yang and Vafai, 2006, 2008; Ai and Vafai, 2006). The lumen domain is considered as a cylindrical geometry with radius of R₀ (310 μm) and axial length L (0.2232 m) surrounded by the porous layers of endothelium, intima, IEL, and media with their detailed information given in Table 2a (Karner et al., 2001; Prosi et al., 2005; Yang and Vafai, 2006, 2008; Ai and Vafai, 2006; Chung and Vafai, 2012). It should be noted that intima properties given in this table are only used for validation with previous works, as it will be based on fiber matrix theory for later results. FIGS. 1A and 1B illustrate schematic diagrams of configuration for endothelium, intima, IEL, and intima fiber matrix 100 and arterial wall 150 respectively.

FIG. 1C illustrates schematic diagram of configuration for analyzed domain 160 for a healthy artery (Yang and Vafai, 2006; Ai and Vafai, 2006).

### 1.2. Atherosclerotic Plaque and Stenosis

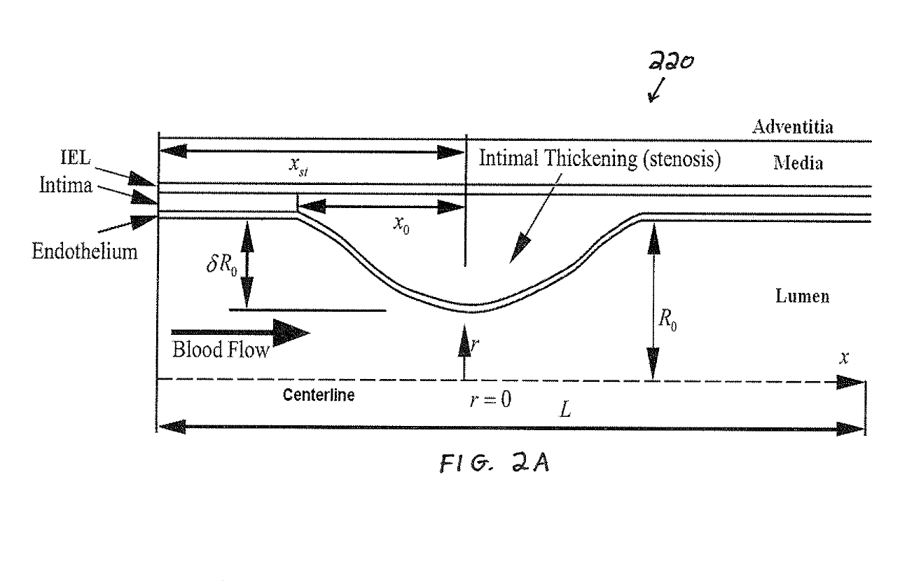

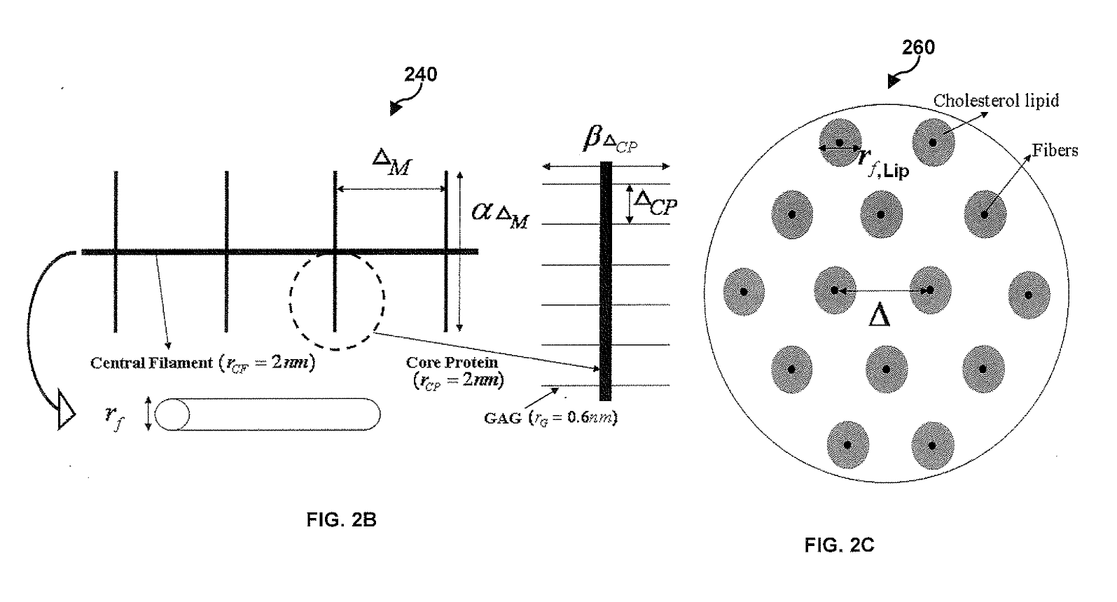

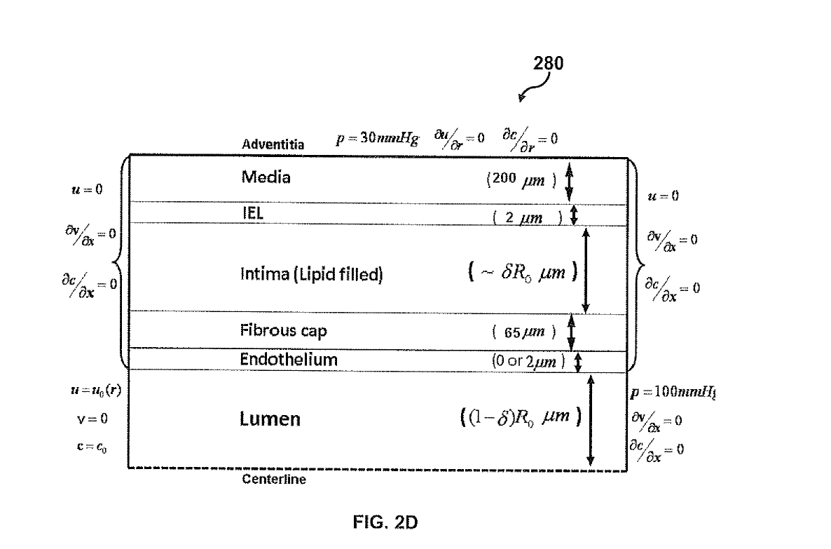

To study LDL transport inside a diseased artery, a computational domain 220 similar to that used in Ai and Vafai's (2006) work is utilized as shown in FIG. 2A. FIGS. 2B and 2C illustrate schematic diagrams of configuration 240 and 260 for proteoglycan fibers within intima (Huang et al. 1994) and fiber matrix filled with cholesterol lipid, respectively, in accordance with the disclosed embodiments. The atherosclerotic plaque is considered by a partial wall thickening within the intima layer that causes stenosis, characterized by δ, ratio of maximum thickness to lumen radius, xₛₜ, its axial location from inlet, and x₀, its half width. However, Ai and Vafai (2006) considered the transport properties of all arterial layers within a normal artery when cholesterol lipid accumulation is not considered. In this work, the lipid filling effect is brought in by applying the fiber matrix theory within a computational domain 280 that incorporates the multi-layered structure of the diseased arterial wall as shown in FIG. 2D.

### 1.3. Governing Equations

A steady state assumption is invoked based on the negligible effect of blood pulsation (Yang and Vafai, 2006; Chung and Vafai, 2012). The hydraulic and molecular transport in the lumen region is described by conservation of mass, momentum and species as:

∇·\vec{u}=0

−∇p+μ_{f}∇²\vec{u}=0

\vec{u}·∇c=D_{f}∇²c Eq. (1)

where \vec{u} is the velocity vector, c LDL concentration, p hydraulic pressure, and μ_{f} and D_{f} are the plasma viscosity and diffusivity coefficient respectively.

The flow and mass transfer governing equations within the four layers, endothelium, intima, IEL, and media, can be represented by Darcy-Brinkman equation while incorporating the Staverman-Kedem-Katchalsky membrane equation (Kedem and Katchalsky, 1958):

$$∇ \cdot {u}^{->} = 0 \\ - ∇ p + {μ}_{eff} {∇}^{2} {u}^{->} - \frac{{μ}_{eff}}{K} {u}^{->} = 0 \\ ( 1 - σ ) {u}^{->} \cdot ∇ c = {D}_{eff} {∇}^{2} c - kc \qquad Eq . ( 2 )$$

where μ_{eff} is the effective fluid viscosity, K hydraulic permeability; a reflection coefficient; D_{eff} effective LDL diffusivity; k reaction coefficient which is 3.197×10⁻⁴ [s¹] inside the media layer and zero in the other layers (Prosi et al., 2005; Yang and Vafai, 2006, 2008).

The property values for each of the layers are listed in Table 2a, while the variable intima properties due to the lipid accumulation are considered later in this work. The flow and molecular transport characteristics within the layers under the influence of lipid accumulation such as the fibrous cap are also described by Equation 2 and the corresponding properties are given in Table 2b.

### 1.4. Boundary Conditions

The boundary conditions 160 are illustrated in FIG. 1C, where the axial velocity u at the entrance is considered to have a fully developed profile u₀(r) expressed by:

u₀=U₀(1−(r/R₀)²) at x=0, 0≦r≦R₀ Eq. (3)

where the maximum entrance velocity U₀ is taken as 0.338 m/s (Yang and Vafai, 2006; Karner et al., 2001) and LDL concentration at the entrance c₀ is taken as 28.6×10⁻³ mol/m³ (Katz, 1985; Tarbell, 1993; Yang and Vafai, 2006).

Hydraulic pressure p is set to be fixed at the outlet of lumen and the outer surface (media-adventitia interface) with the values of 100 mmHg and 30 mmHg resulting in a total pressure drop of 700 mmHg through the arterial wall (Meyer et al., 1996; Yang and Vafai, 2006). Continuity conditions for the flow and mass transfer are invoked at the interface between each of the layers while incorporating the Staverman filtration condition (Yang and Vafai, 2006; Chung and Vafai, 2012) as:

$$[ ( 1 - σ ) v c - {D}_{eff} \frac{\partial c}{\partial r} ] {\vert}_{+} = [ ( 1 - σ ) v c - {D}_{eff} \frac{\partial c}{\partial r} ] {\vert}_{-} \qquad Eq . ( 4 )$$

where v is the filtration velocity of the blood flow penetrating through the arterial wall in the radial direction.

### 1.5. Fiber Matrix Model and Intima Properties

The intima is mainly formed by proteoglycan fibers (FIG. 2), and looser-thicker collagen fibers (Frank and Fogelman, 1989), which can be represented as a homogeneous fiber matrix 100 as shown in FIG. 1A. Compared to endothelium and IEL, Ai and Vafai (2006) pointed out that diffusion in intima layer is not substantial, which was also confirmed in Yang and Vafai's (2006) study. The micro-structure of intima fiber matrix can be characterized by its porosity ε and effective fiber radius r_{f}. A common way to calculate the effective radius of intima protein, r_{f} (Huang et al., 1994; Dabagh et al., 2009) is:

$${r}_{f} = {[ \frac{α {r}_{M}^{2} + {r}_{CF}^{2}}{α + 1} ]}^{1 / 2} \qquad Eq . ( 5 a )$$

where α is the length ratio of proteoglycan monomers to central filament, with a value which is variant between 3 to 10 (Lark et al., 1988), r_{CF} is radius of central filament with a value around 2 nm (Buckwalter and Rosenberg, 1982), and r_{M} is the effective monomer radius calculated by:

r_{M}=[βr_{G}²+r_{CP}²]^{1/2} Eq. (5b)

where β is the length ratio of glycosaminoglycan (GAG) fiber to protein core with a value which is variant between 5 to 10 (Lark et al., 1988), r_{G} is radius of GAG with a value of 0.6 nm, r_{CP} is radius of proteoglycan core protein with a value of 2 nm (Buckwalter and Rosenberg, 1982). By taking α=3 and β=5 (Dabagh et al., 2009; Liu et al. 2011), we can obtain the effective fiber radius for proteoglycan as 2.31 nm.

Utilizing the Carman-Kozney equation (Curry and Michel, 1980; Curry, 1984a, b), the intima's permeability can calculated as:

$$K = \frac{{r}_{f}^{2} {•}^{3}}{4 {G ( 1 •• )}^{2}} \qquad Eq . ( 6 a )$$

where ε is the porosity of intima, and G is the Kozney constant which, for randomly oriented fibers, is calculated as (Happel and Brenner, 1965):

$$G = \frac{2}{3} \frac{2 {•}^{3}}{( 1 •• ) [ 2 ln ( \frac{1}{1 ••} ) •3 + 4 ( 1 •• ) {• ( 1 •• )}^{2} ]} + \frac{1}{3} \frac{2 {•}^{3}}{( 1 •• ) [ ln ( \frac{1}{1 ••} ) • \frac{ 1 {• ( 1 •• )}^{2}}{1 + {( 1 •• )}^{2}} ]} \qquad Eq . ( 6 b )$$

The molecular transport properties for LDL particle through intima, such as the effective diffusivity D_{eff} and reflection coefficient σ can be calculated by (Huang et al. 1992, 1994):

$$\begin{matrix}{D}_{eff} = {D}_{f} exp [ - {( 1 - ɛ )}^{1 / 2} ( 1 + \frac{{r}_{m}}{{r}_{f}} ) ] & Eq . ( 7 a ) \\ σ = {( 1 - φ )}^{2} & Eq . ( 7 b )\end{matrix}$$

where rₘ is LDL molecular radius taken as 11 nm (Huang et al., 1992, 1994), and φ is the partition coefficient obtained by:

$$φ = exp [ - ( 1 - ɛ ) ( \frac{2 {r}_{m}}{{r}_{f}} + \frac{{r}_{m}^{2}}{{r}_{f}^{2}} ) ] \qquad Eq . ( 7 c )$$

In the work of Dabagh et al. (2009) and Liu et al. (2011), in addition to proteoglycan fibers, the collagen fibers are also considered. As such the transport properties were calculated as:

$$\begin{matrix} \frac{1}{K} = \frac{1}{{K}_{PG}} + \frac{1}{{K}_{CG}} & Eq . ( 8 a ) \\ {D}_{eff} = {D}_{f} [ - {( 1 - {ɛ}_{PG} )}^{1 / 2} ( 1 + \frac{{r}_{m}}{{r}_{f}} ) ] ( {ɛ}_{PG} + {ɛ}_{CG} - 1 ) exp [ - {( 1 - {ɛ}_{CG} )}^{0.5} ( 1 + \frac{{r}_{m}}{{r}_{CG}} ) ] & Eq . ( 8 b ) \\ σ = {( 1 - φ )}^{2} & Eq . ( 8 c ) \\ φ = exp [ - ( 1 - {ɛ}_{PG} ) ( \frac{2 {r}_{m}}{{r}_{f}} + \frac{{r}_{m}^{2}}{{r}_{f}^{2}} ) ] ( {ɛ}_{PG} + {ɛ}_{CG} - 1 ) exp [ - {( 1 - {ɛ}_{CG} )}^{0.5} ( 1 + \frac{{r}_{m}}{{r}_{CG}} ) ] & Eq . ( 8 d )\end{matrix}$$

where ε_{PG} and ε_{CG} are the porosity of proteoglycan and collagen fibers, and r_{CG} is radius of collagen fiber set as 20 nm (Dabagh et al, 2009). Also, K_{PG} and K_{CG} are calculated through Equations 6a and 6b, using ε_{PG} and ε_{CG} as the porosity and r_{f} and r_{CG} as the fiber radius. However, due to a much coarser distribution of collagen fibers, it is considered to have an insignificant impact. Therefore, an alternative way is to use Equations 6 and 7 with porosity defined by ε=ε_{PG}ε_{CG} (Dabagh, 2009).

### 2. COMPARISONS

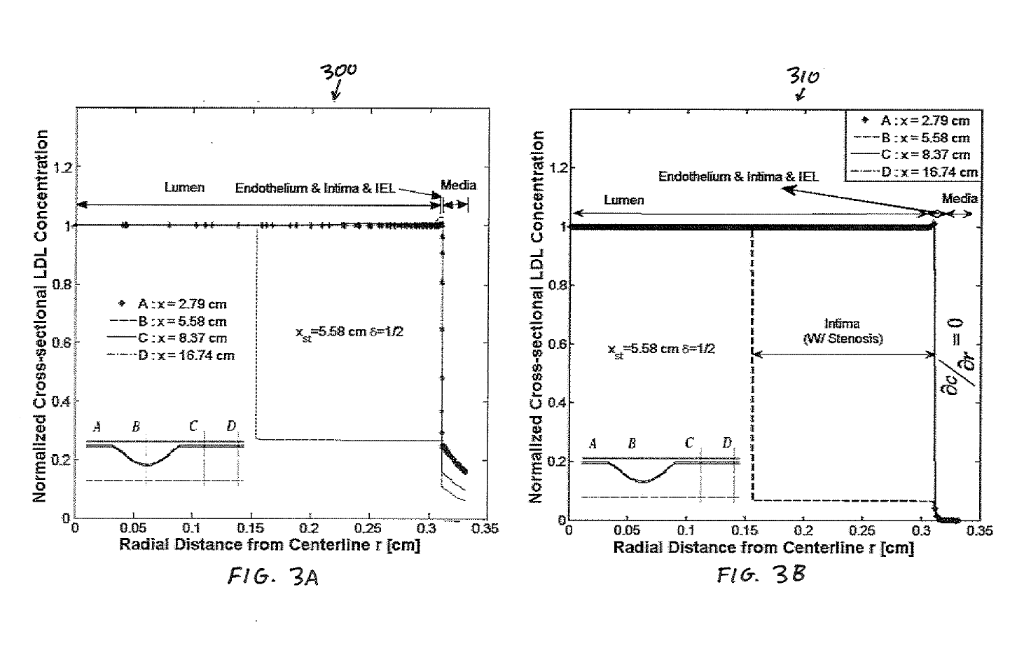

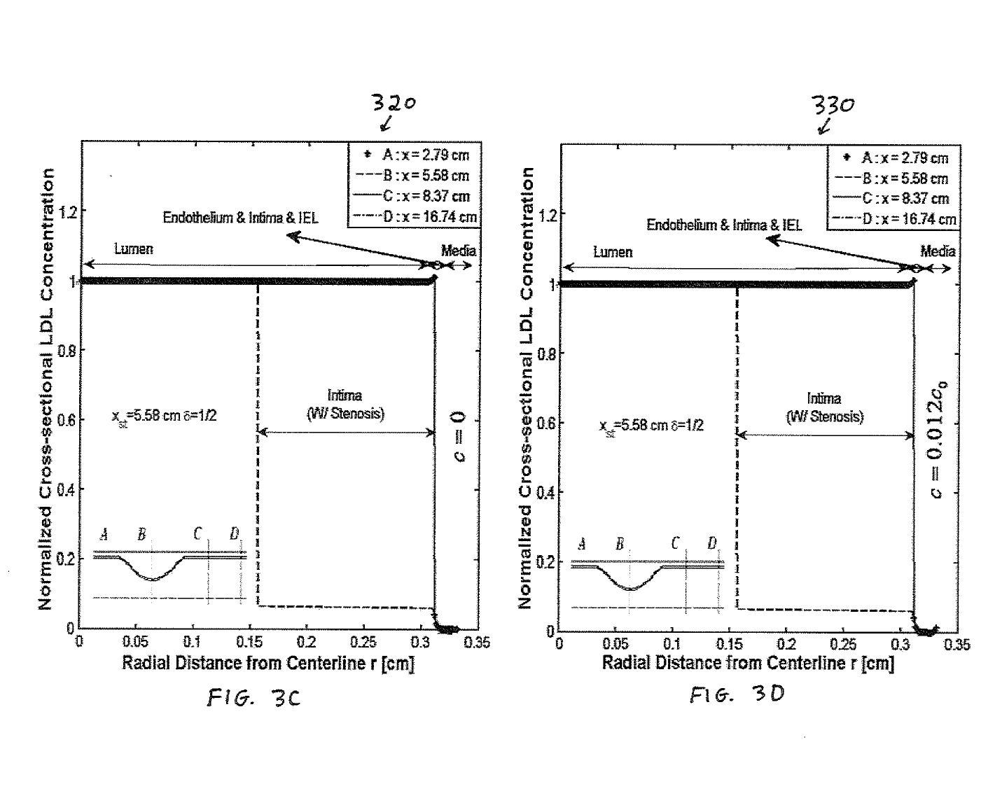

FIGS. 3A-3B illustrate graphs 300 and 310 showing a normalized LDL concentration c/c₀ across diseased artery layers in the presence of stenosis with δ=0.5 and x₀=2R₀ for comparison with Ai and Vafai (2006) work. FIGS. 3C-3D illustrate graphs 320 and 330 showing a normalized LDL concentration c/c₀ across diseased artery layers in the presence of stenosis with δ=0.5 and x₀=2R₀ for two different boundary conditions at the media-adventitia interface (r=3.314 mm) c=0 and c=0.012c₀.

The results for the velocity field and mass concentration are obtained numerically with relative and absolute errors less than 10⁻³ and 10⁻⁶, respectively. The model developed in this work is compared with the work of Ai and Vafai (2006) for both normal artery [presented in Chung and Vafai (2012)] and diseased artery with stenosis, using a different solution methodology. The results from both of the cited works shows an insignificant impact as a result of either thickening of the wall or different stenosis locations (FIGS. 3A-3B), even with different boundary conditions on the outer surface (adventitia side, r=3.314 mm) that is commonly applied (FIGS. 3C-3D). The methodology utilized in the current study in analyzing LDL transport inside an artery was validated in the work of Chung and Vafai (2012).

### 3. RESULTS AND DISCUSSION

### 3.1. Sensitivity Study on Plaque Geometry and Model Simplification

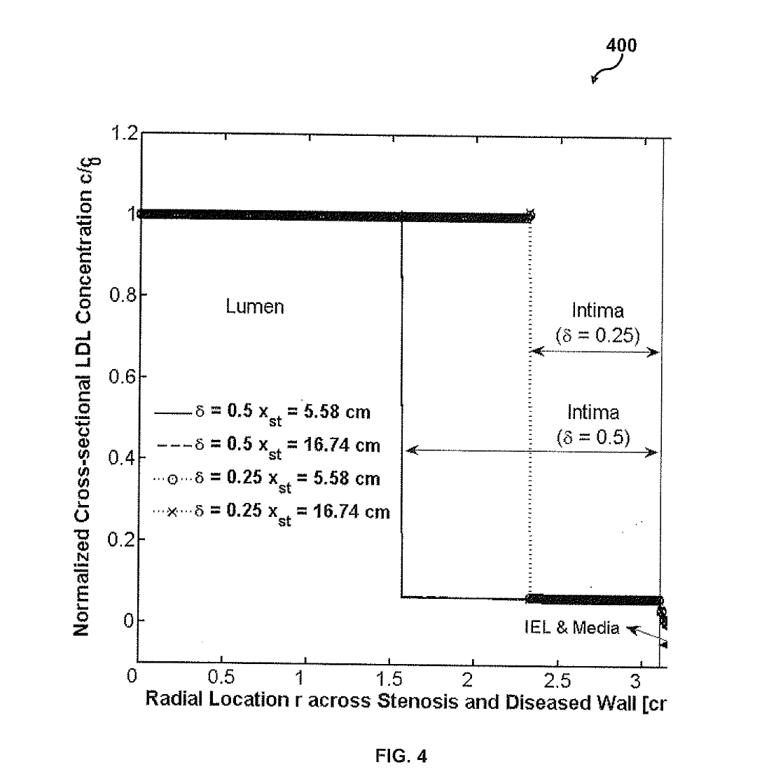

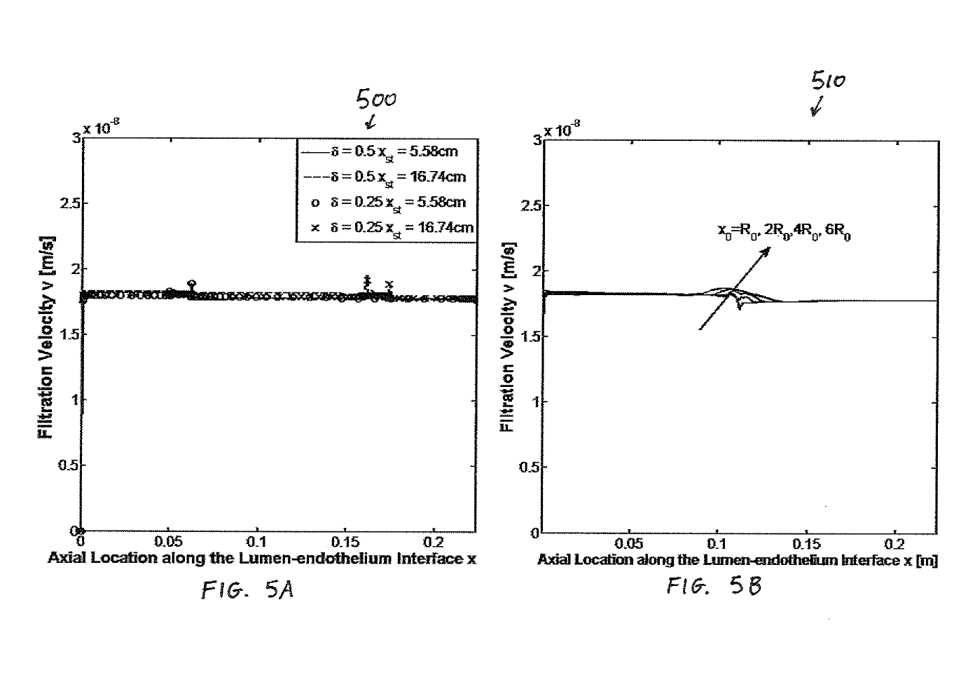

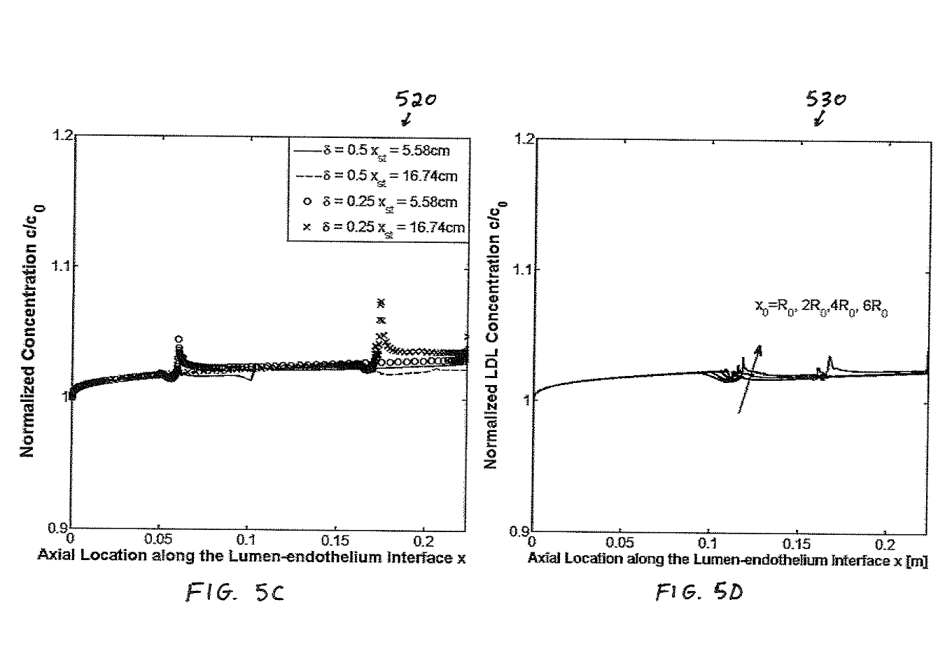

Various researchers have modeled the artery both with and without atherosclerosis; however, mostly modeling it as a wall-free or simplified-wall model, which doesn't allow them to look into the highly pertinent transport behavior within the arterial wall. In FIG. 4, depicted in graph 400, the impact of the macro-structure, i.e., the shape of stenosis due to atherosclerosis is shown to be negligible. FIGS. 5A-5B illustrate graphs 500 and 510 showing filtration velocity along the lumen-endothelium interface for different wall thickening ratio δ and stenosis locations xₛₜ, and different values of the plaque half widths x₀, respectively. FIGS. 5C-5D illustrate graphs 520 and 530 showing LDL concentration along the lumen-endothelium interface for different wall thickening ratio δ and stenosis locations xₛₜ and different values of the plaque half widths x₀, respectively. FIGS. 5A-5D, which zooms into the lumen-wall interface for pressure, filtration velocity, and LDL concentration, still shows that this effect is negligible.

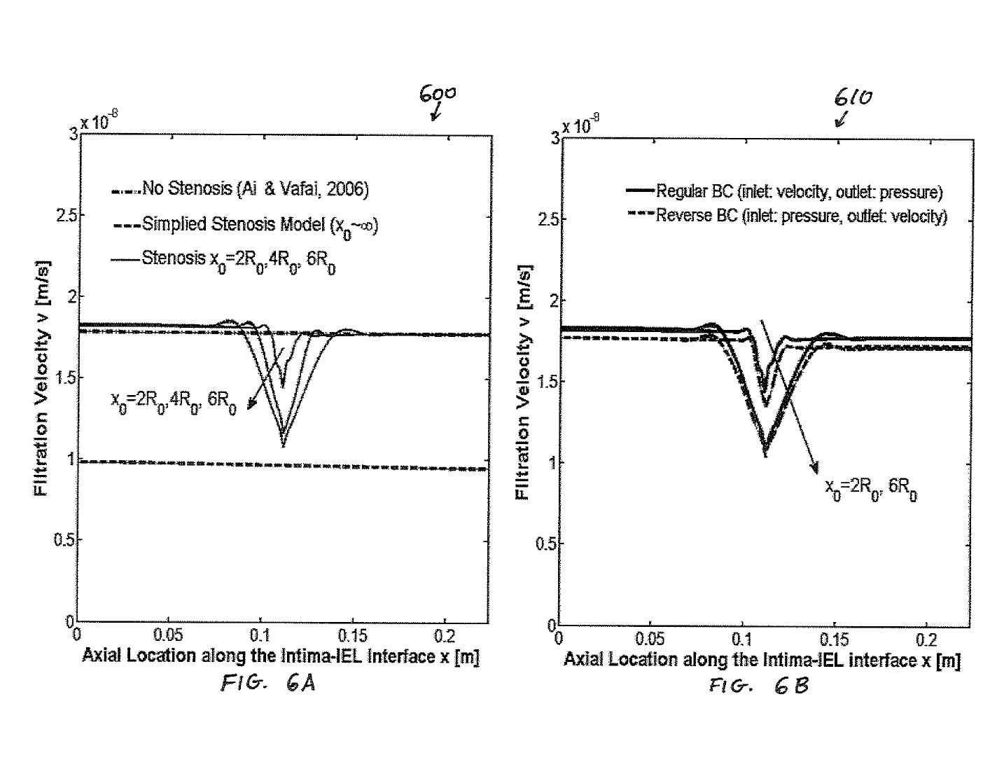

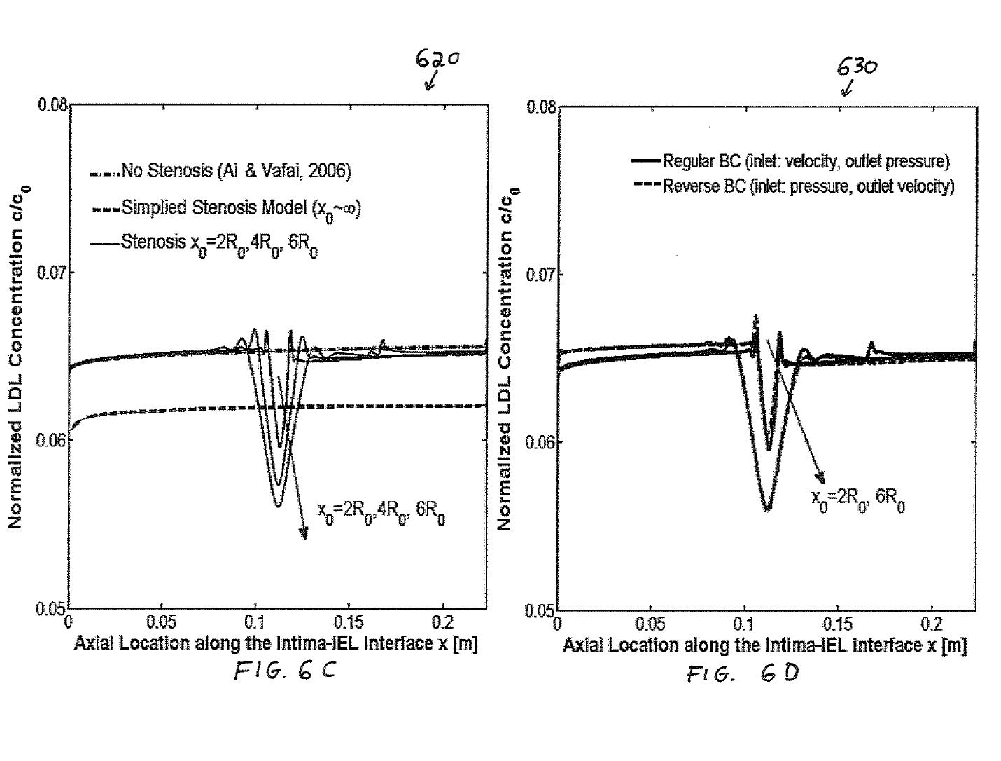

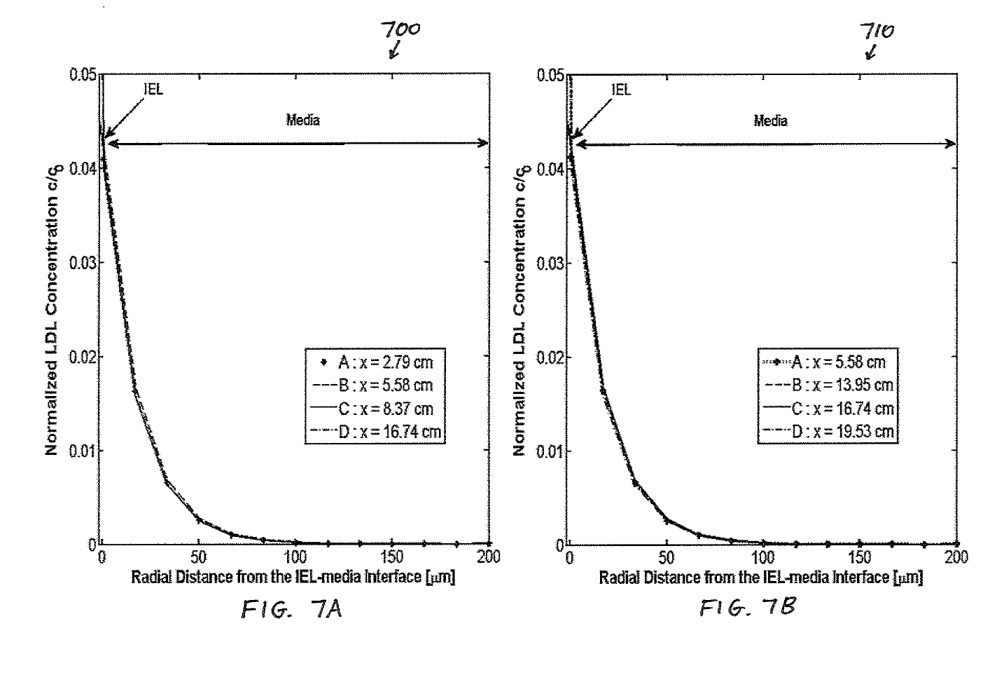

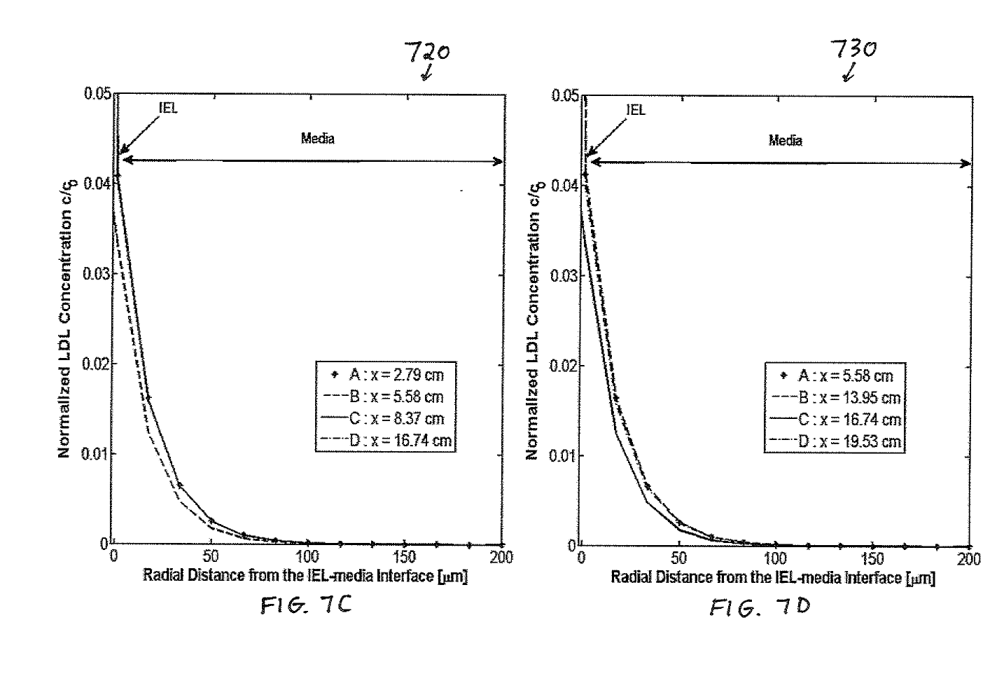

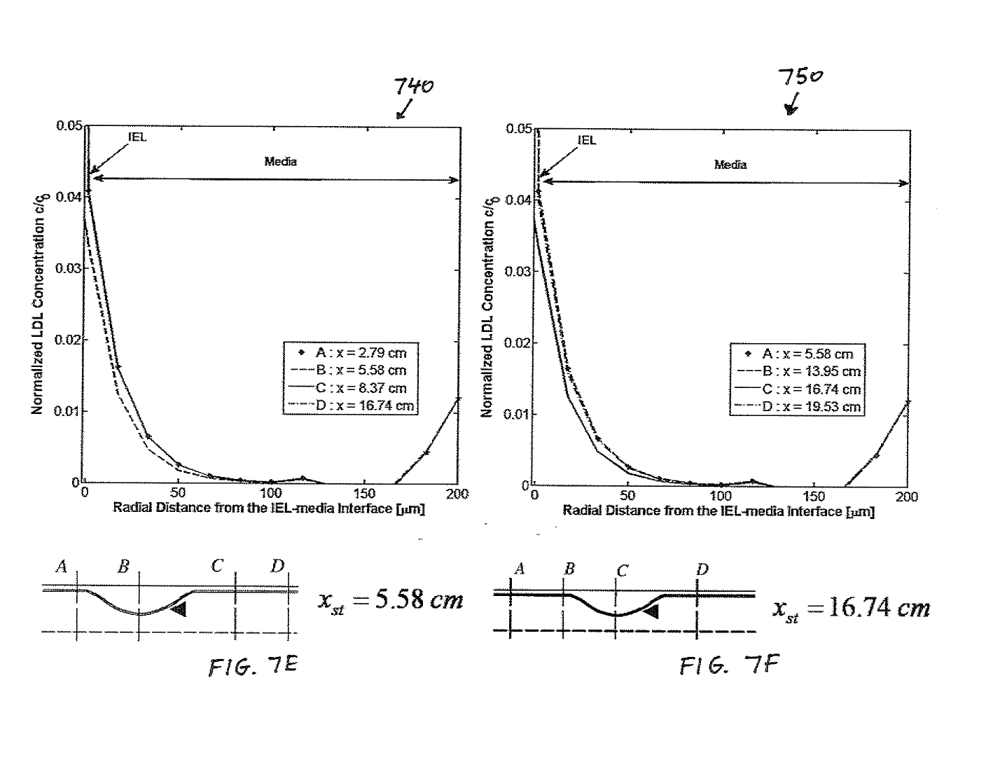

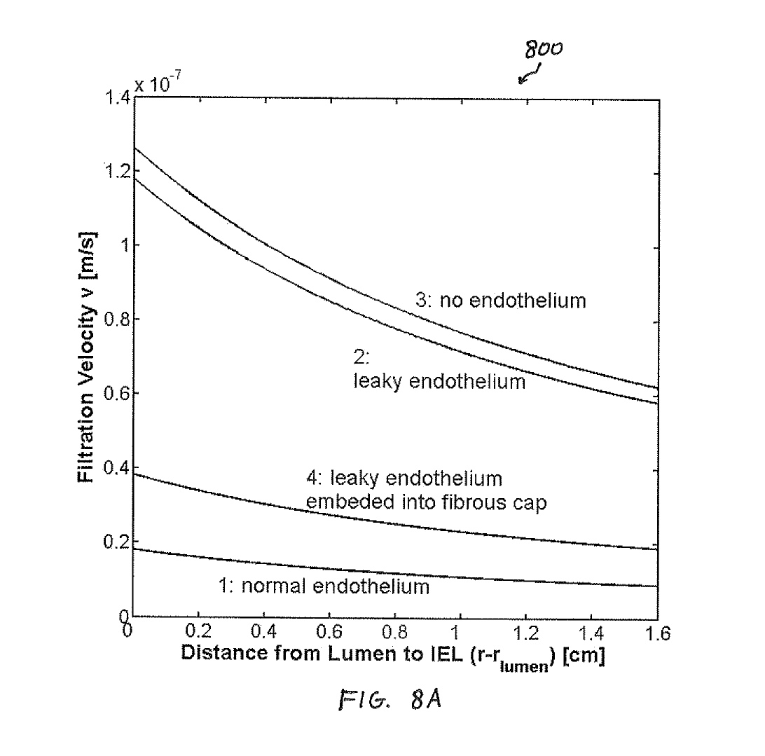

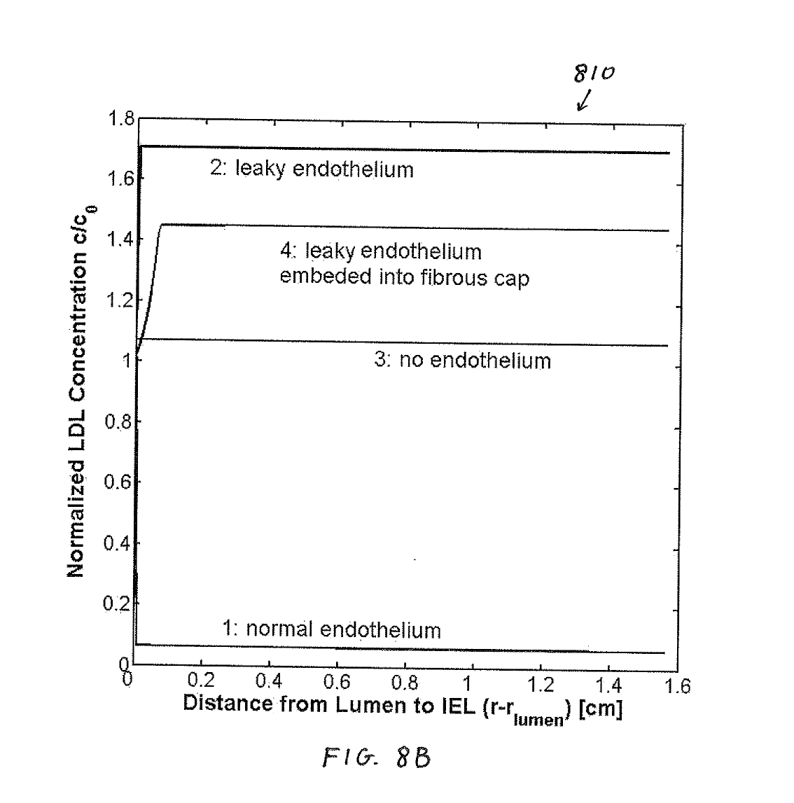

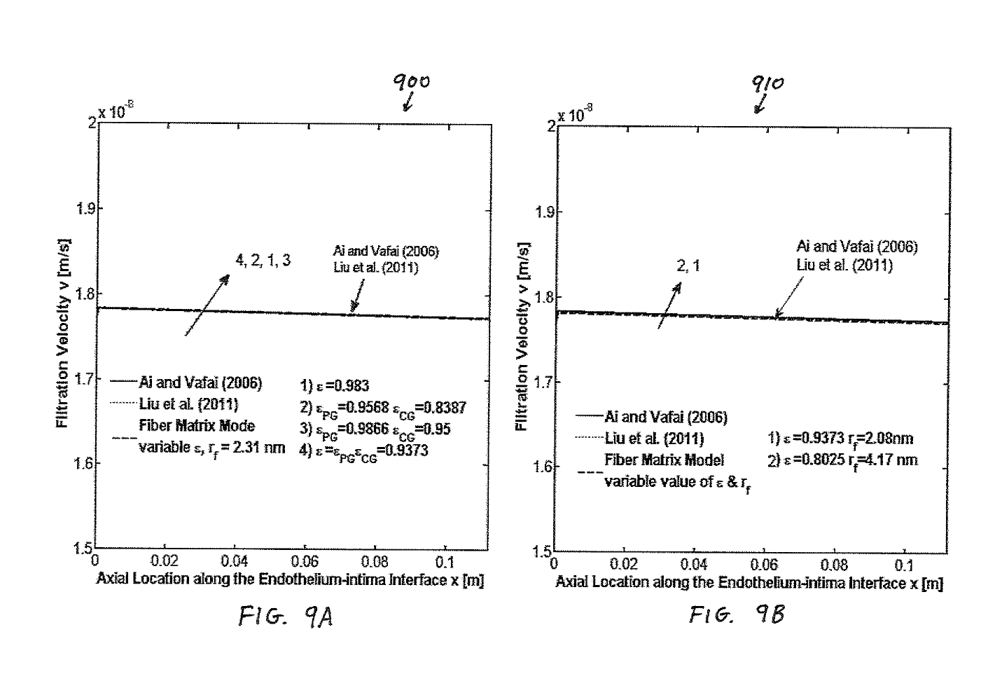

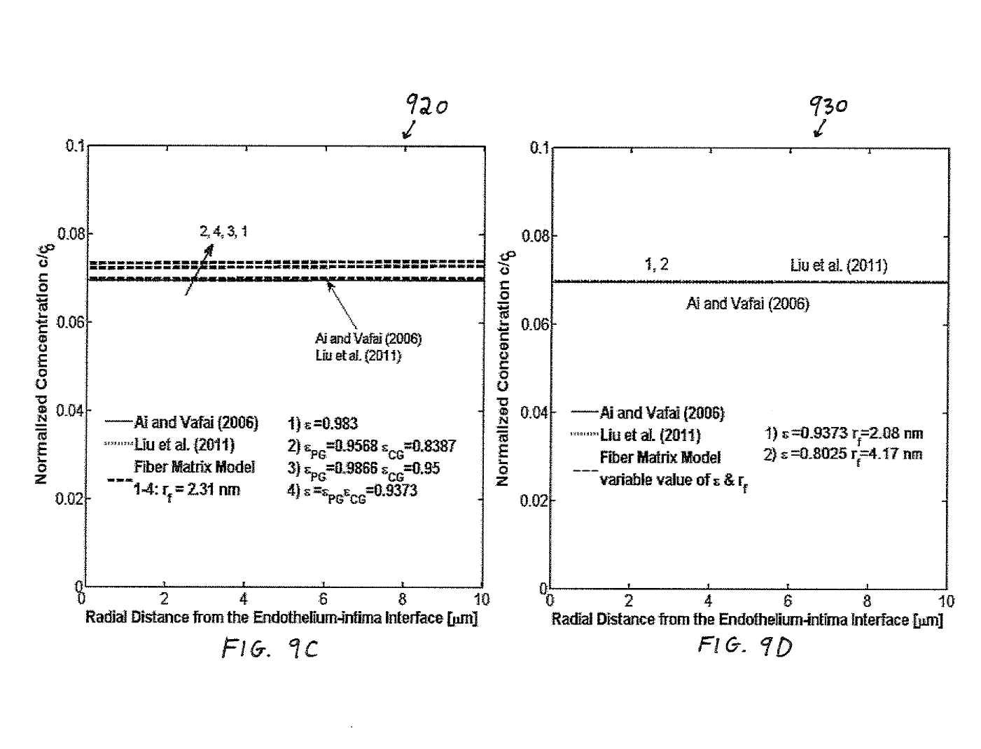

On the other hand, the impact of the atherosclerosis is shown to be present within the intima as shown in FIGS. 6A-6D due to a smaller exposed surface area of the wall to lumen caused by the radial wall thickening. Along the intima-IEL interface, the pressure, filtration velocity, and LDL concentration display a significant drop as seen in FIGS. 6A and 6C, graphs 600 and 620. Furthermore, this drop becomes more pronounced as the plaque builds up. FIGS. 6B and 6D shows graphs 610 and 630 that using the reverse boundary conditions at the inlet (specifying pressure instead of velocity) and outlet (specifying velocity instead of pressure) displays the same phenomena seen in FIGS. 6A and 6C, and confirms the fact that the edge effects are insignificant. FIGS. 7A-7F illustrate graphs 700, 710, 720, 730, 740, and 750 showing normalized LDL concentration c/c₀ across IEL and media of a diseased artery at different locations x in the presence of stenosis with δ=0.5, xₛₜ=5.58 cm, and 16.74 cm for boundary condition at the media-adventitia interface (r=3.314 mm) as ∂c/∂r=0, c=0, c===0.012c₀. However, this impact diminishes further inside the IEL and intima layers shown in FIGS. 7A-7F, which shows the effect of boundary condition is also negligible. Therefore, one can conclude that the axial location has a negligible effect on the results.

In conclusion, the atherosclerotic impact at the lumen-wall interface is shown to be minor, compared to the concentration distribution within each different layer of the wall. The present results show that the thickening of the arterial wall impacts plasma and LDL transport in the radial direction substantially more than in the axial direction. As such, a simplified computational domain shown in FIG. 2D can be applied. On the other hand, the microstructure and the transport properties of the arterial layers are impacted by atherosclerosis either by cholesterol lipid accumulation and tissue hyperplasia, or the dysfunction of endothelial layer, which is discussed in detail in the present work.

### 3.2. Dysfunctional Endothelium and Fibrous Cap

Dysfunctional endothelium and fibrous cap forms as LDL cholesterol deposits accumulate within intima (Hossain et al., 2011). The transport properties of normal junction (Ai and Vafai, 2006) and leaky junction endothelium (Curry, 1984a; Chung and Vafai, 2012), as well as effective diffusivity of fibrous cap (Hossain et al., 2011) are listed in Table 2b. Graphs 800 and 810 in FIGS. 8A-8B shows comparison between results obtained for dysfunctional endothelium and fibrous cap on penetration of blood and LDL. The comparison shows that dysfunctional endothelium causes a deduction of the resistance from lumen into wall, while fibrous cap results an increasing resistance.

### 3.3. Calculation of Intima Properties Through Fiber Matrix Model

Using healthy intima (no cholesterol/lipid accumulation) microstructure characteristics, effective fiber radius r_{f} and porosity ε are used as a reference point, the transport property values are obtained based on the microstructure information through the fiber matrix method. Table 3a lists intima properties obtained from Equation 6 and 7 (considering only the proteoglycan fiber), or Equation 8 (considering both proteoglycan and collagen fibers) with effective fiber radius r_{f} obtained from Equation 5 as 2.31 nm, and the porosity taken from the following prior works: 1) E=0.983 (Karner et al., 2001; Yang and Vafai, 2006); 2) ε_{PG}=0.9568 and ε_{CG}=0.8387 (Dabagh et al., 2009; Liu et al., 2011); 3) ε_{PG}=0.9866 and ε_{CG}=0.95 (Dabagh et al. 2009); 4) ε=ε_{PG}ε_{CG}=0.9373 (Dabagh et al. 2009).

FIGS. 9A and 9C illustrates graphs 900 and 920 the filtration velocity and LDL concentration along the endothelium-intima interface utilizing the data given in Table 3a, compared with the works of Ai and Vafai (2006) and Liu et al. (2011) given in Table 3c. As can be seen in FIGS. 9A and 9C, even though the intima property values are quite different, as seen in Table 2, the impact on plasma and LDL molecular transport is limited.

Ai and Vafai (2006) pointed out that, within the intima layer, the transport is mostly dominated by convection flux, and their analytical work resulted a reflection coefficient σ of 0.8292. As such, from the results given in Table 3a, case 2 (ε=ε_{PG}ε_{CG}=0.8025) and case 4 (ε=0.9397) are selected for comparison with the results of Ai and Vafai (2006). Utilizing Equations 7b and c, with the intima reflection coefficient of 0.8292 and porosities ε of 0.9373 and 0.8025, results in an effective radius of intima protein fiber r_{f} as 2.08 and 4.17, respectively [nm]. These are represented in Table 3b based on Equations 6 and 7.

In FIGS. 9B and 9D, the graphs 910 and 930 shows the comparisons for both filtration velocity and LDL concentration at the endothelium-intima interface using the data given in Table 3b. A perfect agreement is seen with the results of Ai and Vafai (2006) and Liu et al. (2011). It should be noted that since a fiber radius of 2.08 nm is closer to the value which is obtained through Equation 5, which is also utilized in Yang and Vafai's work (2006), it is more reasonable to assign the porosity ε and effective fiber radius r_{f} for a healthy intima as 0.9373 and 2.08 nm.

### 3.4. Impact of Variations in Intima Properties by Lipid Accumulation

The structure of intima for a normal artery is shown in FIG. 1A, while during LDL molecule accumulation, the structure resembles the schematic shown in FIG. 2C displaying a thicker fiber radius r_{f,Lip} and a lower porosity ε_{Lip} due to the lipid deposits as compared to a healthy intima (ε and r_{f}). As discussed earlier, the normal intima porosity ε and the effective fiber radius r_{f} are set as 0.9397 and 2.08 nm. The maximum fiber thickening ratio (r_{f,Lip}/r_{f}) is set as 150 which is the ratio of the thickness of atherosclerotic plaque with δ=0.5 to the thickness of a normal intima layer. With the effective intima porosity ε_{Lip} and protein fiber thickening rate r_{f,Lip}/r_{f} varying between 20-93.97% and 1-150, respectively, the variable properties for the hydraulic permeability K, effective diffusivity D_{eff}, and reflection coefficient σ are obtained through fiber matrix model using Equations 6 and 7.

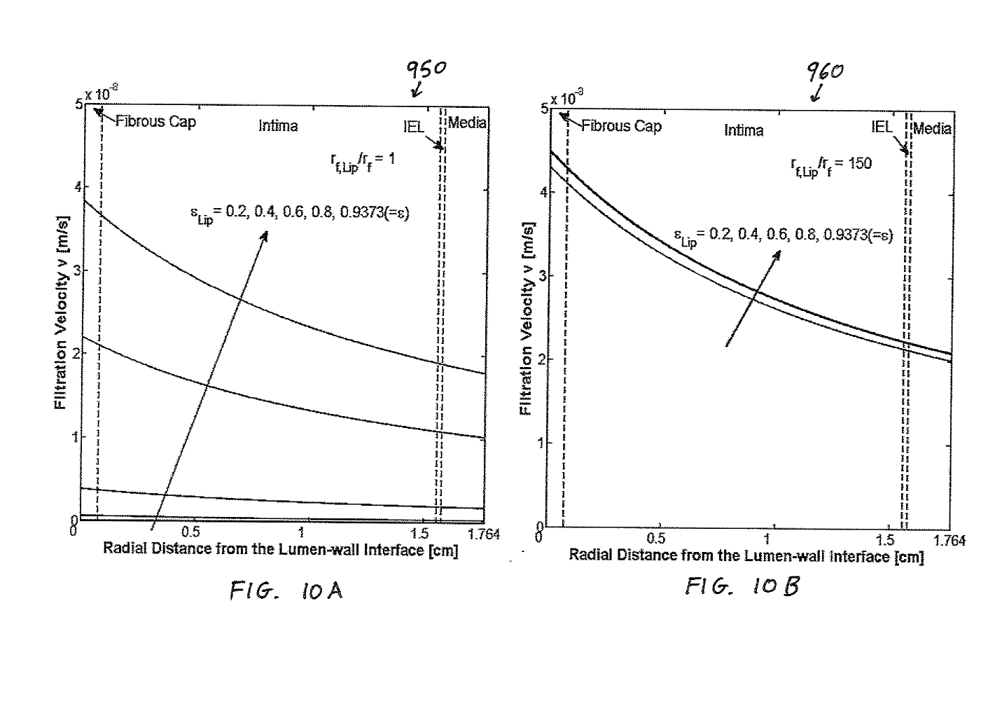

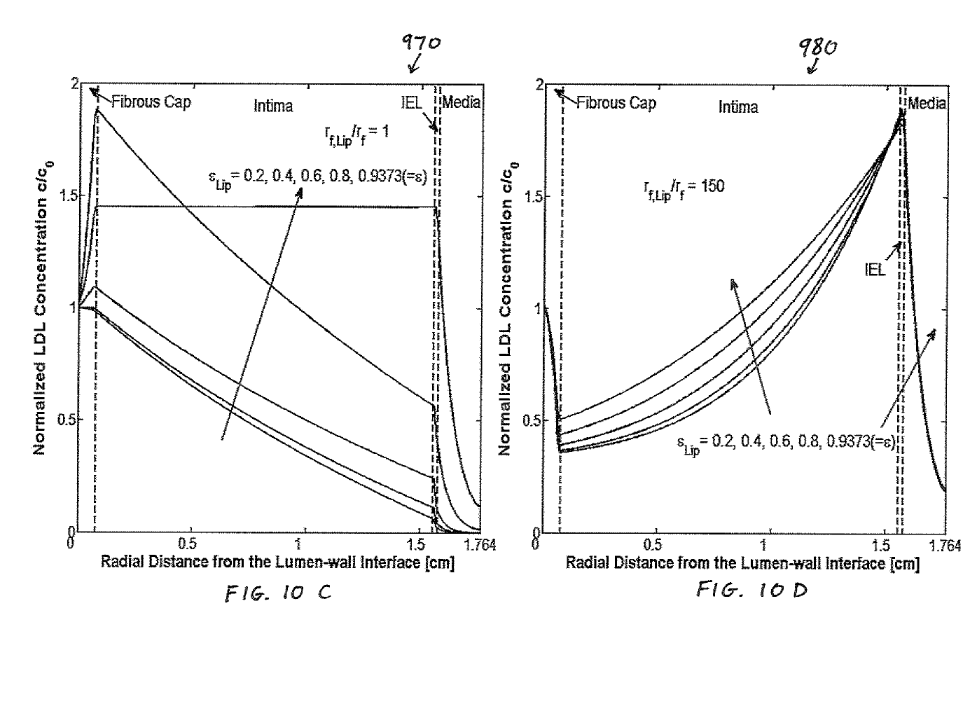

To describe LDL transport within a diseased artery (FIG. 2D), a fibrous cap with an embedded dysfunctional endothelium (model 4 in FIGS. 8A-8B) is selected to represent the transport properties. FIGS. 10A and 10B illustrates the graphs 950 and 960 showing impact of variable properties due to lipid accumulation on filtration velocity subject to different effective intima porosity ε_{Lip} and protein fiber thickening ratio r_{f,Lip}/r_{f}. As can be seen in FIGS. 10A and 10B, a lower porosity leads to a lower permeability, while a thicker effective fiber radius reduces this impact by providing a larger space between the fibers.

FIGS. 10C and 10D displays graphs 970 and 980 showing the effect of variable properties on the LDL transport. As expected, a higher porosity results in more LDL molecule deposits inside the arterial wall. As the thickening ratio r_{f,Lip}/r_{f} increases, a larger space between the fibers is created resulting in a reduction in the selective behavior for LDL particles inside the intima due to formation of stenosis. As such, LDL particles are deposited more near the intima-IEL interface, instead of the inner wall surface, because the IEL layer with its higher rate of the particle selection takes over the role of blocking LDL particle migration from the lumen side.

### 4. CONCLUSIONS

Our model clearly demonstrates in detail how cholesterol lipid caused by molecular accumulation affects the microstructure, as well as the LDL transport properties, in each of the arterial layers, which lead to dysfunctional arterial wall and stenosis that result atherosclerotic cardiovascular disease. Applying the model and the results developed in this study, one can more easily understand the initiation and development of atherosclerosis affected by LDL transport, and further explore improvements on early diagnosis and treatment of atherosclerosis and other related cardiovascular diseases.

The microstructure details and characteristics of the endothelium and intima due to the formation of plaque/stenosis are incorporated into the present analysis. Pertinent scenarios for transport through a dysfunctional endothelium and fibrous cap within intima are invoked. The variable intima properties affected by LDL molecule accumulation are analyzed, and its impact on the hydraulic and molecular transport in a thickened arterial wall is examined. Lower porosity by lipid blockage results in a lower permeability, which is diminished by thickening of effective fiber due to more space between the fibers as a result of stenosis.

It will be appreciated that variations of the above disclosed apparatus and other features and functions, or alternatives thereof, may be desirably combined into many other different systems or applications. Also, various presently unforeseen or unanticipated alternatives, modifications, variations or improvements therein may be subsequently made by those skilled in the art which are also intended to be encompassed by the following claims.

What is claimed is:

1. A method for analyzing Low-Density Lipoprotein (LDL) transport within a multi-layered arterial wall comprising:
utilizing a multi-layered model to represent heterogeneity using Darcy-Brinkman and Staverman filtration equations to describe transport within porous layers of a wall;
utilizing a fiber matrix model to represent cholesterol lipid accumulation and resulting variable properties; and
obtaining reference values of intima's porosity and effective fiber radius through said fiber matrix model, wherein said fiber matrix model characterizes micro-structure within intima.

2. The method of claim 1 further comprising analyzing transport through dysfunctional endothelium and fibrous cap, and the impact on hydraulic and molecular transport properties by LDL accumulation in a thickened arterial wall.

3. The method of claim 1 further comprising analyzing effect of variable properties on plasma and LDL molecular transport.

4. The method of claim 1 further comprising analyzing variable intima properties affected by LDL molecule accumulation and examining its impact on the hydraulic and molecular transport in a thickened arterial wall.

5. The method of claim 1 wherein lower porosity by lipid blockage results in a lower permeability, which is diminished by thickening of effective fiber due to more space between the fibers as a result of stenosis.
